(19) United States
(12) Patent Application Publication
(10) Pub. No.: US 2021/0189020 A1
(43) Pub. Date: Jun. 24, 2021

(54) DANDELION RUBBER PRODUCTION BY THERMAL CYCLES IMPLEMENTATION

(71) Applicants: Association pour les Transferts de Technologies du Mans, LE MANS (FR); INSTITUT NATIONAL DE LA RECHERCHE AGRONOMIQUE (INRA), PARIS CEDEX 07 (FR); THE OHIO STATE UNIVERSITY, COLOMBUS, OH (US); UNIVERSITE D'ANGERS, ANGERS (FR); Institut National Supérieur des Sciences, RENNES (FR)

(72) Inventors: Julien BOSSELUT, ANGERS (FR); Soulaiman SAKR, ANGERS (FR); Camille LE BRAS, LA MEIGNANNE (FR); Katrina CORNISH, WOOSTER, OH (US)

(21) Appl. No.: 16/723,018

(22) Filed: Dec. 20, 2019

(51) Int. Cl. C08C 1/04; C08H 8/00; A01H 6/00

(52) U.S. Cl. CPC C08C 1/04; A01H 6/00; C08H 8/00

(57) ABSTRACT

The present invention concerns a method for culturing a plant, said method comprising a culture step wherein said plant is submitted to repeated thermal cycles, wherein, for each thermal cycle, cool temperature and warm temperature are alternately artificially applied to all or a part of said plant, over a period shorter than natural seasons.

The present invention also concerns a method for producing natural rubber from latex-producing plants, said method comprising the steps of (a) culturing the latex-producing plant by implementing the method of culture of the invention, (b) harvesting part or all of root part of said plant, and (c) extracting natural rubber from the root part harvested at step b).

The present invention concerns the culture of plants, in particular of latex-producing plants.

The global market of natural rubber represents 14 millions of tons per year. Europe, which represents 11% of the global market, aims at reaching 20% of self-supply in order to reduce its dependency towards hevea natural rubber, which is today mainly produced in Asian countries.

Several European and American research teams work on Kazakh dandelion (Taraxacum kok-saghyz) rubber. The technical quality of this type of natural rubber has been confirmed by several tire manufacturers. However, the current culture techniques of Kazakh dandelion plants do not enable rubber to be produced in an economically viable way.

One way of making the Kazakh dandelion rubber production economically viable is to increase the synthesis of rubber by the plant, either by selecting or developing plant varieties producing high levels of polyisoprene as disclosed in Stolze et al. (2017) Plant Biotechnology Journal 15:740-753, or by adapting the culture conditions of the plant to increase the rubber synthesis level.

There is thus a need for new agronomic engineering enabling optimization of the production of high-value secondary metabolites, in particular carbonaceous polymers, by plants, in particular dandelion plants.

The present invention meets this need.

The present invention results from the unexpected finding by the inventors that by alternating repeatedly warm culture temperature and cool culture temperature during the culture of the plant, in particular in soil-less conditions, it was possible to increase the production yield of rubber by the plant. The production yield was particularly optimized when warm culture temperature was applied during daytime and cool culture temperature was applied during nighttime, preferably only to the root part of the plant.

The present invention thus concerns a method for culturing a plant, said method comprising a culture step wherein said plant is submitted to repeated thermal cycles, wherein, for each thermal cycle, cool temperature and warm temperature are alternately artificially applied to all or a part of said plant, over a period shorter than natural seasons.

The present invention also concerns a method for producing natural rubber from latex-producing plants, said method comprising the steps of:

a) culturing the latex-producing plant by implementing the method of culture of the invention,

b) harvesting the root part of said plant, and

c) extracting natural rubber from the root part harvested at step b).

### DETAILED DESCRIPTION OF THE INVENTION

### Plant

By “plant”, is meant herein a multicellular photosynthetic eukaryote of the kingdom Plantae.

In a particular embodiment, said plant is a land plant.

By “part of plant” is notably meant herein the aerial parts of a plant, stems, branches, leaves, fruit, seeds and/or flowers, and/or the below-ground parts (or root parts) such as rhizomes, roots and/or bulbs.

The method of the invention is particularly useful for optimally producing natural rubber from latex-producing plants.

Accordingly, in a particular embodiment, said plant is a latex-producing plant. In a more particular embodiment, said plant is a Taraxacum kok-saghyz dandelion or a Taraxacum brevicorniculatum dandelion.

### Thermal Cycles

By “thermal cycle” is meant herein the alternation of different culture temperatures, in particular two culture temperatures, on a given period.

In the context of the present invention, for each thermal cycle submitted to the plant, cool temperature and warm temperature are alternately artificially applied to all or a part of the plant, over a period shorter than natural seasons.

By “natural season” is meant herein a division of the year marked by changes in weather, ecology and the amount of daylight. Natural seasons are typically spring, summer, autumn and winter. A natural season lasts typically 3 months.

Accordingly, in a particular embodiment, said alternation of cool and warm temperatures is applied over a period of less than 3 months.

In a particular embodiment, said alternation of cool and warm temperatures is applied over a period of a month or of less than a month.

In another particular embodiment, said alternation of cool and warm temperatures is applied over a period of a week or of less than a week.

In another particular embodiment, said alternation of cool and warm temperatures is applied over a period of a day.

In a particular embodiment, the cultured plant may be simultaneously submitted to a photoperiodism, i.e. a daytime/nighttime alternation.

By “daytime” is meant herein a period during which the plant is submitted to solar or artificial radiation.

By “nighttime” is meant herein a period during which the plant is not submitted to any solar or artificial radiation.

Accordingly, in a particular embodiment, said alternation of cool and warm temperatures is applied over a period during which the plant is submitted to a daytime (solar or artificial radiation)/nighttime (no radiation) alternation.

In this particular embodiment, the plant is preferably submitted, in particular over a day, to cool temperature during the nighttime period and to warm temperature during the daytime period. In said particular embodiment, the day is a 24-hour day.

Preferably, the photoperiodism daytime/nighttime is between 0.2 and 5, preferably between 0.5 and 2.

In a particularly preferred embodiment, the plant is submitted, over a 24-hour day, to cool temperature during the nighttime period and to warm temperature during the daytime period, the photoperiodism daytime/nighttime being between 0.2 and 5, preferably between 0.5 and 2.

By “cool temperature” is meant herein a temperature lower than room temperature, typically a temperature lower than or equal to 15° C., preferably higher than 6° C.

By “warm temperature” is meant herein a temperature similar to or higher than room temperature, typically a temperature strictly higher than 15° C., preferably lower than 45° C.

In a particular embodiment, the difference between the cool temperature and the warm temperature is of at least 4° C.

In a preferred embodiment, the warm temperature is strictly higher than 15° C., preferably higher than 17° C., and preferably lower than 45° C., more preferably lower than 30° C. In a particularly preferred embodiment, the warm temperature is comprised between 18° C. and 22° C.

In a preferred embodiment, the cool temperature is lower than or equal to 15° C., preferably lower than 13° C., and preferably higher than 6° C., more preferably higher than 10° C. In a particularly preferred embodiment, the cool temperature is comprised between 12° C. and 13° C.

In a particularly preferred embodiment, the warm temperature is comprised between 18° C. and 22° C. and the cool temperature is comprised between 12° C. and 13° C.

In the context of the invention, cool temperature and warm temperature are artificially applied to all or a part of said plant.

By “artificially” is meant herein that the alternation of cool and warm temperatures is not due to a natural phenomenon, in particular a meteorological phenomenon, but to an engineered event produced by humans.

In a particular embodiment, the cool temperature and the warm temperature are both applied to the whole plant.

In an alternative embodiment, the cool temperature and the warm temperature are only applied to parts of the plant, preferably to different parts of the plant.

In a preferred embodiment, the cool temperature is only applied to the root part of the plant while the aerial part of the plant remains at the warm temperature.

### Plant Culture

In a particular embodiment, the culture step is a soil-less culture step.

By “soil-less culture” is meant herein any method of growing plants without the use of soil as a rooting medium. Soilless culture include hydroponic culture, substrate culture and aeroponic culture.

Accordingly, in a particular embodiment, the soil-less culture step is a hydroponics, aeroponics or substrate culture step.

As well-known from the skilled person, hydroponic culture (or water culture) include deepwater culture, float hydroponics, nutrient film technique and deepflow technique. In hydroponic culture, roots are typically partially or completely dipped in a nutrient solution.

As well-known from the skilled person, substrate culture includes gravel culture, sand culture, bag culture, container culture and trough culture. In substrate culture, the plant is typically culture on an artificial, mineral or organic growing media.

As well-known from the skilled person, aeroponic culture is a process of growing plants in an air or mist environment without the use of soil or an aggregate medium.

In a preferred embodiment, said culture step in a hydroponic culture step, such as a deepwater culture step.

In said particular embodiment, the plant roots are typically placed in a nutrient solution.

By “nutrient solution” is meant herein an aqueous solution comprising mainly inorganic ions from soluble salts of essential elements for plants, and optionally organic compounds. Preferably, said nutrient solution comprises nitrogen, phosphorus and potassium.

In a said particular embodiment, said alternation of cool and warm temperatures is applied to the plant through the nutrient solution.

In a particular embodiment, the cool temperature is applied to the root part of the plant by refrigerating the nutrient solution and the warm temperature is applied to the plant by maintaining the nutrient solution at the warm temperature.

The other culture conditions applied to the plant during the culture step, such as lightening during the daytime period, hygrometry, fertilizers, are preferably conventional culture conditions well-known from the skilled person.

Typically, the culturing step is performed using HPS or LED lightening during the daytime period, 50-80% hygrometry, nutrient solution comprising nitrogen, phosphorus and potassium and weekly foliar application of magnesium-rich biostimulant composition.

In a particular embodiment, the culture step is implemented after a step of seedling growth, preferably a step of 8 to 15 weeks of seedling growth.

By “seedling growth” is meant herein the plant growth starting when germination is completed (i.e. from the emergence of the radicle through the seed coat) until the appearance of enough green leaves to make the plant independent of stored energy.

Preferably, during the seedling growth step, the growing seed is not submitted to thermal cycles as defined in the section “Thermal cycles” above.

In said particular embodiment, the plant obtained after the seedling growth step is repotted to implement the culture step of the invention.

The culture step is thus typically implemented on a mature plant.

Said culture step is preferably implemented, typically on a mature plant, during more than 24 hours, preferably more than a week, still preferably during more than two weeks, most preferably during more than 4 weeks, typically during 1 to 4 weeks.

In a particular embodiment, the method of culture of the invention further comprises, after the culture step, a step of harvesting the cultured plant or a part thereof.

Preferably, all or part of the root part of the plant is harvested during the harvesting step.

Harvesting can be implemented by any technique well-known from the skilled person such as the harvesting technique disclosed in US 2016/0237254. Typically, harvesting can be implemented by cutting roots of the plant, preferably in such a manner that the roots are partly left on the plant, for example by using scissors, a knife or the like.

### Production of Secondary Metabolites

As indicated above, the culture method of the invention enables optimizing the rubber yield production by dandelion plants.

The culture method of the invention is thus particularly useful for optimizing production of a secondary metabolite by said plant.

By “secondary metabolite” is meant herein a organic compound produced by the plant which is not directly involved in the normal growth, development and reproduction of the plant. Secondary metabolites are typically required for interaction of plants with their environment and produced in response to stress.

Plant secondary metabolites typically include flavonoids and allied phenolic and polyphenolic compounds, terpenoids, and nitrogen-containing alkaloids and sulphur-containing compounds.

In a preferred embodiment, said secondary metabolite is a terpenoid.

In a preferred embodiment, said secondary metabolite is a polymer, in particular a terpenoid polymer, more particularly polyisoprene, more preferably cis-1,4-polyisoprene, most preferably high molecular weight cis-1,4-polyisoprene, still preferably cis-1,4-polyisoprene with a molecular weight ranging from 30,000 to 10,000,000.

As well-known to the skilled person, cis-1,4-polyisoprene constitutes more than 99% of natural rubber.

### Method for Producing Natural Rubber

As indicated above, the culture method of the invention enables optimizing the rubber yield production by dandelion plants and can thus be used in production methods of natural rubber.

The present invention thus also concerns a method for producing natural rubber from latex-producing plants, said method comprising the steps of:

a) culturing a latex-producing plant by implementing the method of culture of the invention,

b) harvesting part or all of the root part of said plant, and

c) extracting natural rubber from the root part harvested at step b).

By “natural rubber”, also called “India rubber”, “latex”, “Amazonian rubber” or “caoutchouc”, is meant herein a material consisting of high molecular weight polymers of isoprene, optionally with minor impurities of other organic compounds, and water.

In the context of the invention, natural rubber refers to dandelion rubber.

Step a) of the method of production of the invention consists in culturing a latex-producing plant, as defined in section “Plant” above, by implementing the method of culture defined above.

Step b) of the method of production of the invention consists in harvesting the root part, as defined in section “Plant” above, of said plant.

Harvesting of said root part can be performed by any technique well-known from the skilled person, as described above.

Step c) of the method of the invention consists in extracting natural rubber from the root part harvested at step b).

Extraction of natural rubber from the root part of the plant can be made by any technique well-known to the skilled person. Typically, emulsion oozing out from the harvested portions of the roots may be collected. The oozing emulsion may be collected by any method and, for example, it may be collected as appropriate using a tool such as a spatula. Natural rubber may be extracted by crushing the harvested roots followed by extraction with an organic solvent. Alternatively, natural rubber may be extracted by drying the harvest roots and then separating the biomass from the rubber using an aqueous extraction process.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

The present invention will be further illustrated by the figures and example below.

### BRIEF DESCRIPTION OF THE FIGURES

FIG. 1: Dry root mass (in g) after 10 weeks and 16 weeks of culture with and without refrigeration of the nutrient solution.

### EXAMPLE

The following strategy was used by the inventors:
1) Leading the culture under ideal conditions (in terms of light, photoperiod, temperature, etc. . . . ) for 10 weeks to promote the development of root biomass,
2) After 10 weeks, applying a temperature drop to the root system (via refrigeration of the nutrient solution) at night, in order to induce the biosynthesis of rubber and inulin degradation to sugars (fructose) usable for biosynthesis. During the day, the growing conditions permit normal plant metabolism and promote photosynthetic activity and the capture of carbon.
   Root-accumulated inulin during the daytime when the photosynthesis is active in leaves, may be used for rubber synthesis in cold-treated roots during the night.

### Materials and Methods

### Hydroponic System Design

The hydroponics system able to accommodate plants for further experiments was designed and prototyped between January and October 2018.

The roots being the part of the plant producing rubber, it was important to achieve a hydroponic system facilitating access to them. A first culture system inspired by the method DWC (Deep water culture) has therefore been developed.

“Deep water culture” involves producing plants with a minimum of substrate. Indeed, the roots of the plant are retained by a basket pot (filled with ball clay or rockwool) and soaked directly in the oxygenated nutrient solution.

A first DWC system which was developed is a tank of 400 l (60 cm in height), and a second prototype used for the experiment, was developed in a 100 l tank (35 cm in height). To this tank was added a stirring system of the nutrient solution (pump 1500 l/h). Circular bubblers of 35 cm in diameter were placed at the bottom of the tank and were fed by an air compressor (2700 l/h). A PVC plate was pierced to the diameter of the pots, each ferry welcoming between 12 and 25 plants

### Refrigeration System of the Nutrient Solution

In order to reduce the temperature of the nutrient solution, a cold group (capacity 250 l) was added to the culture system. The 1500 l/h pump immersed in the tank was then connected to the cold group. A 130 μm filter was added before the cold unit to prevent the system from clogging.

The pump ran 24 h/24 h, so the nutrient solution was constantly circulating in the DWC tank. The cold unit was set using a timer on the time zone desired.

### Plant Material

Taraxacum kok-saghyz plants used during the experiment were derived from seeds provided by the Ohio State University.

### Refrigeration Test of the Nutrient Solution

The plants were grown hydroponically in DWC system for 4 months (16 weeks) in accordance with the culture conditions described in Table 1 below.

TABLE 1
Culture conditions of Taraxacum kok-saghyz in DWS hydroponics

| | Week 1-4 | Week 5-10 (culture | Week 11-16 |
|---|---|---|---|
| | (greenhouse) | room) | (greenhouse) |
| Air | 18-23° C. day/ | 20° C. day/15° C. | 18-23° C. day/13- |
| temperature | 13-18° C. night | night | 18° C. night |
| Photoperiod | 16 h day/8 h night | | |
| Lighting | HPS + natural | 4000K light LED | HPS + natural light |
| | light | 300-350 μmol | (150-300 μmol) |
| | (150-300 μmol) | | |
| Hygrometry | 80-100% | 70% | 50-80% |
| Nutritional | Week 1: | Week 5-10: | Week 11-16: |
| solutions | Clear water | N15/P10/K30 | N15/P10/K30 |
| | unadjusted pH | pH = 5.8-6.2 | pH = 5.5-6 |
| | EC = 0.4 mS | EC = 1-1.2 mS | EC = 1 mS |
| | Week 2: N10/ | Foliar application 2 | Foliar application |
| | P52/K10 | times per week of | 1 time per week of |
| | pH = 5.5-6 | biostimulant | biostimulant |
| | EC = 0.6 mS | enriched in | enriched in |
| | Week 3-4: N10/ | Magnesium | Magnesium |
| | P52/K10 | (Phylgreen ® Mg) | (Phylgreen ® Mg) |
| | pH = 5.5-6 | | |
| | EC = 0.8 mS | | |

Seeds were germinated on a honeycombed plate of rock wool covered with a thin layer of vermiculite (<1 cm). The plate was then placed under high relative humidity for 3 weeks in a greenhouse.

After 4 weeks of cultivation, the seedlings were repotted in a basket pot on clay balls and 72 plants were placed in culture chamber for 6 weeks.

After 10 weeks of culture, the roots of 12 plants were harvested, lyophilized and then evaluated for root dry mass and rubber concentration. Other plants were then moved to a greenhouse where the test of chilling the nutrient solution was performed.

For this test two batches of 7 randomly selected plants were used: the first batch was placed in a control condition, where the nutrient solution was not refrigerated (its temperature varied between 18-22° C.). The second batch was placed in refrigerated condition (the temperature of the nutrient solution was lowered to 12-13° C. overnight).

In hydroponics, the ideal temperature of the nutrient solution is between 16 and 20° C. This temperature ensures both good root development and good oxygen level of the nutrient solution. Below 15° C., plants are under stress. However, this stress does not have a lasting physiological impact if it is not prolonged or if the temperature does not fall below a certain threshold.

The plants were grown for 6 weeks, then the roots were harvested to assess the root dry mass and rubber concentration

### Extraction of Sugars, Resins, and Rubber

After lyophilization, the roots were mixed and milled with a ball mill. The protocol used for the extraction of sugars, resin and rubber was an ASE method (Accelerated Solvent Extraction) disclosed in Ramirez-Cadavid et al. (2018) Industrial crops and products 122:647-656.

Extractions were performed in duplicate on each pool of individuals.

### Results

### Evaluation of the Root Biomass

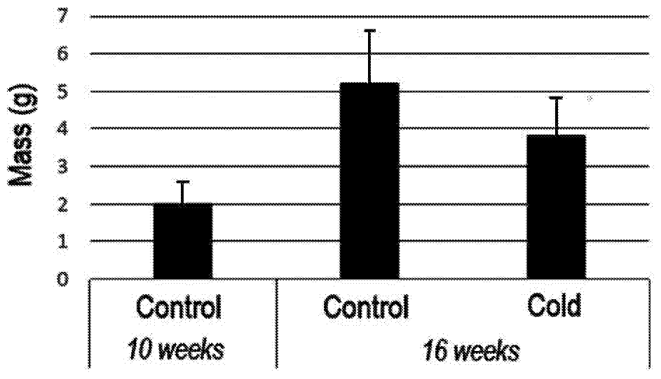

The plants cultivated for 16 weeks had a higher dry biomass than those taken after 10 weeks of culture (see FIG. 1).

After 16 weeks, the dry root mass was 5.23 g/plant (FIG. 1) for individuals grown in control condition in the non-refrigerated nutrient solution.

Cold-processed plants tended to present lower root biomass (3.84 g/plant).

The cold applied at night did not seem to prevent the development of root biomass but the difference in biomass after 6 weeks of treatment could be explained by a slower metabolism caused by the cold.

### Evaluation of the Rubber Concentration

TABLE 2
Concentration of sugars, resin and rubber (mg/g dry root)

| | | Amount (mg/g dry root) | | |
|---|---|---|---|---|
| Culture duration | Treatment | Total sugars | Resin | Rubber |
| 10 weeks | Control | 605 ± 28 | 39.0 ± 1.4 | 7.0 ± 2.1 |
| 16 weeks | Control | 702 ± 18 | 29.5 ± 3.5 | 17.5 ± 7.7 |
| | Cold | 650 ± 35 | 28.0 ± 0.2 | 42.0 ± 25.4 |

Extractions were performed in duplicate on each pool of individuals.

With the exception of the resin, the concentrations of sugars and rubber were higher after 16 culture weeks (Table 2).

Cold-grown plants had a lower sugar concentration than plants cultivated in a control condition after 16 weeks. Conversely, the plants grown in the chilled solution had a higher rubber concentration (on average 2.4 times higher). Nevertheless, after 16 weeks, the concentrations of sugars in plants grown in the chilled nutrient solution were higher than those obtained after 10 weeks of culture, which showed that bringing cold at night did not block the plant's sugar metabolism.

### CONCLUSION

This example demonstrates the efficacy of cold treatment during cultivation for increasing the rubber concentration in Taraxacum kok-saghyz.

1. Method for culturing a plant, said method comprising a culture step wherein said plant is submitted to repeated thermal cycles wherein, for each thermal cycle, cool temperature and warm temperature are alternately artificially applied to all or a part of said plant, over a period shorter than natural seasons.

2. Method according to claim 1, wherein said alternation of cool and warm temperatures is applied over a period of a day.

3. Method according to claim 1, wherein said alternation of cool and warm temperatures is applied over a period during which the plant is submitted to a daytime (solar or artificial radiation)/nighttime (no radiation) alternation.

4. Method according to claim 3, wherein the plant is daily submitted to cool temperature during the nighttime (no radiation) period and to warm temperature during the daytime (solar or artificial radiation) period.

5. Method according to claim 4, wherein the day is a 24 hour-day.

6. Method according to claim 3, wherein the photoperiodism daytime/nighttime is between 0.2 and 5.

7. Method according to claim 1, wherein the difference between the cool temperature and the warm temperature is of at least 4° C.

8. Method according to claim 1, wherein the warm temperature is strictly higher than 15° C.

9. Method according to claim 1, wherein the cool temperature is lower than or equal to 15° C.

10. Method according to claim 1, wherein the cool temperature is only applied to the root part of the plant while the aerial part of the plant remains at the warm temperature.

11. Method according to claim 1, wherein the culture step is a soil-less culture step.

12. Method according to claim 1, for optimizing production of a secondary metabolite by said plant.

13. Method according to claim 12, wherein said secondary metabolite is a polymer.

14. Method according to claim 13, wherein said polymer is cis-1,4-polyisoprene.

15. Method according to claim 1, wherein said plant is a latex-producing plant.

16. Method according to claim 15, wherein said plant is a Taraxacum kok-saghyz dandelion or a Taraxacum brevicorniculatum dandelion.

17. Method according to claim 1, wherein said culture step is implemented after a step of 8 to 15 weeks of seedling growth.

18. Method according to claim 17, wherein the culture step is implemented on a mature plant during more than 24 hours.

19. Method according to claim 1, further comprising, after the culture step, a step of harvesting the cultured plant or a part thereof.

20. A method for producing natural rubber from latex-producing plants, said method comprising the steps of:
a) culturing the latex-producing plant by implementing the method of culture according to claim 15,
b) harvesting part or all of the root part of said plant, and
c) extracting natural rubber from the root part harvested at step b).
